(19) United States
(12) Patent Application Publication
(10) Pub. No.: US 2007/0198043 A1
(43) Pub. Date: Aug. 23, 2007

(54) Bone marrow aspiration device

(76) Inventors: Daniel L. Cox, Palo Alto, CA (US); Eugene T. Michal, San Francisco, CA (US); Robert D. Ainsworth, Scotts Valley, CA (US); Gregory W. Chan, San Francisco, CA (US); Jeong S. Lee, Diamond Bar, CA (US); Albert K. Chin, Palo Alto, CA (US); Geoff Willis, Mountain View, CA (US); Philip S. Yip, Santa Clara, CA (US)

Correspondence Address: BLAKELY SOKOLOFF TAYLOR & ZAFMAN, 1279 OAKMEAD PARKWAY, SUNNYVALE, CA, 94085-4040, US

(21) Appl. No.: 11/360,324

(22) Filed: Feb. 22, 2006

(51) Int. Cl. A61B 17/34; A61M 5/32; A61B 10/00

(52) U.S. Cl. 606/185; 604/272; 600/564

(57) ABSTRACT

A bone marrow aspiration device is described. In one embodiment, the bone marrow aspiration device includes a central body portion having a proximal end and a distal end, and an outer shaft portion coupled to the distal end of the central body portion. The outer shaft portion also has a distal opening. A first lumen is formed by the central body portion and the outer shaft portion, and the first lumen extends from the proximal end to the distal opening. An aspiration needle is disposed within the first lumen. The aspiration needle has a substantially linear configuration when positioned within the first lumen, and a substantially curved configuration when extended from the distal opening. The aspiration needle is adapted to aspirate liquid bone marrow from a first region of a bone cavity.

### TECHNICAL FIELD

Embodiments of the present invention relate to the field of cell processing, and in one particular embodiment, related to repairing cardiac tissue with autologous mononuclear cells obtained from bone marrow.

### BACKGROUND

Autologous transplantation of bone marrow cells into infarcted myocardium shortly after acute myocardial infarction (AMI) has been shown to be a therapy beneficial for improving long-term outcome in these patients. Studies have suggested approximately 40 ml of aspirated bone marrow is required from the patient hours before the transplantation procedure.

The bone marrow is the tissue that manufactures the blood cells and is in the hollow part of most bones. The bone marrow is located in the central region of the bone and has a liquid-like texture and consistency. The bone marrow is surrounded by spongy bone material and a hard bone material such as the cortex. For treatment of AMI patients, bone marrow is typically taken from the hip bone (i.e., iliac crest). Bone marrow aspiration/biopsy procedures are painful and often poorly tolerated in adults. The aspiration procedure typically involves introducing an aspiration needle through the various layers of bone material including the cortex and into the bone marrow of the iliac crest, as illustrated in FIG. 1. Suction is applied through a syringe to remove liquid bone marrow. The aspiration needle is inserted beneath the skin and rotated until it penetrates the cortex, or outer covering of the bone. At least half a teaspoon of marrow is withdrawn from the bone by a syringe attached to the needle. The patient may experience discomfort when the needle is inserted or when the marrow is aspirated. If more marrow is needed, the needle is repositioned slightly, a new syringe is attached, and a second sample is taken. The tip of the needle must be moved after about 5 ml of aspirant to access additional bone marrow in the region and this movement can increase the pain and trauma to the insertion site and increase the time of the aspiration procedure.

### SUMMARY

Embodiments of a device to aspirate or extract bone marrow tissue from a patient are described. The bone marrow aspiration devices described herein facilitate the therapy of autologous bone marrow transplantation in AMI patients by reducing pain associated with the aspiration procedure, improving the efficiency, and tailoring the procedure to the specific requirements of the therapy. In one particular embodiment, the bone marrow aspiration device includes a first outer shaft with a distal cutting tip or edge for penetrating the bone cortex, a proximal handle, and an inner curved, elastic needle with a proximal adaptor suitable for connecting to a syringe. The curved, elastic needle may be made of a shape memory metal such as nickel titanium (NiTi) or another type of super-elastic metal.

In another embodiment of the present invention, the bone marrow aspiration device includes an aspiration needle with a resilient or shape memory wire (e.g., super elastic NiTi wire) disposed within a lumen of the aspiration needle. When the distal end of the wire is advanced out of the aspiration needle, the curved portion may be used to agitate and disturb the surrounding bone marrow region. This facilitates the aspiration of liquid bone marrow through the aspiration needle.

In another embodiment of the present invention, the bone marrow aspiration device may include a tubular anchoring structure, an aspiration needle which may be disposed within a lumen of the anchoring structure, and a mechanism of engagement between the anchoring structure and the aspiration needle which controls the forward movement of the needle into the bone cortex.

Still, another embodiment of the present invention, a method for bone marrow aspiration comprises inserting into a bone cortex a device. This device includes at least: a central body having a proximal and a distal end, and an outer shaft portion coupled to the distal end of the central body portion, with the outer shaft portion having a distal opening. There is at least one of a bone penetration needle and an aspiration needle disposed within a first lumen. The aspiration needle having a substantially linear configuration when positioned within the first lumen, and a substantially curved configuration when extended from the distal opening. The aspiration needle adapts to aspirate liquid bone marrow from a first region of a bone cavity, penetrating the bone cortex using at least one of a bone penetration needle and a cutting tip or edge of a first outer shaft of an aspiration device; and aspirating bone marrow using at least one of a bone penetration needle and the aspiration needle. Further in this method, aspiration may be accomplished by suction via syringe attached near the proximal end of the flexible aspiration needle and/or the bone penetration needle. Aspiration may be performed at different regions of the bone cavity repeatedly by rotating the aspiration needle and moving the aspiration needle distally along a longitudinal axis of the aspiration device. Moreover, a resilient wire, which can be driven by a motorized driving mechanism, may be used to break down bone marrow tissue after initial aspiration of bone marrow from a region to assist in repeated aspirations.

Additional embodiments, features and advantages of the medical device will be apparent from the accompanying drawings, and from the detailed description that follows below.

### BRIEF DESCRIPTION OF THE DRAWINGS

The present disclosure is illustrated by way of example, and not limitation, in the figures of the accompanying drawings in which:

FIG. 1 illustrates a typical procedure for aspirating bone marrow, with a needle inserted through the cortex.

FIG. 2A illustrates one embodiment of a bone marrow aspiration device positioned over a bone structure.

FIG. 2B illustrates the bone marrow aspiration device of FIG. 2A penetrating the bone cortex.

FIG. 2C illustrates an aspiration needle from the bone marrow aspiration device of FIG. 2A advancing through a distal end of an outer shaft portion and into the cavity of bone structure.

FIG. 2D illustrates the aspiration needle from the bone marrow aspiration device of FIG. 2A rotating to a region of the bone cavity.

FIG. 3A illustrates a sectional view of a bone marrow aspiration device in a first configuration.

FIG. 3B illustrates a sectional view of the bone marrow aspiration device in a second configuration.

FIG. 4 illustrates another sectional view of the bone marrow aspiration device of FIG. 3A.

FIG. 5 illustrates a cross-sectional view of the device of FIG. 4 taken along line A-A.

FIG. 6 illustrates a cross-sectional view of the device of FIG. 4 taken along line B-B.

FIG. 7 illustrates a sectional side view of another embodiment of a bone marrow aspiration device.

FIG. 8 illustrates one embodiment of a distal region for the device of FIG. 7.

FIG. 9 illustrates another embodiment of a distal region for the device of FIG. 7.

FIG. 10 illustrates another embodiment of a distal region for the device of FIG. 7.

FIG. 11A illustrates another embodiment of a bone marrow aspiration device positioned over a partially illustrated bone structure.

FIG. 11B illustrates the bone marrow aspiration device of FIG. 11A engaging the surface of the bone cortex.

FIG. 12 illustrates a sectional view of a bone marrow aspiration device.

FIG. 13 illustrates a cross-sectional view of the bone marrow aspiration device of FIG. 12 taken along line A-A.

FIG. 14 illustrates a cross-sectional view of the bone marrow aspiration device of FIG. 12 taken along line B-B.

FIG. 15 illustrates one embodiment of anchoring members.

FIG. 16 illustrates another embodiment of anchoring members.

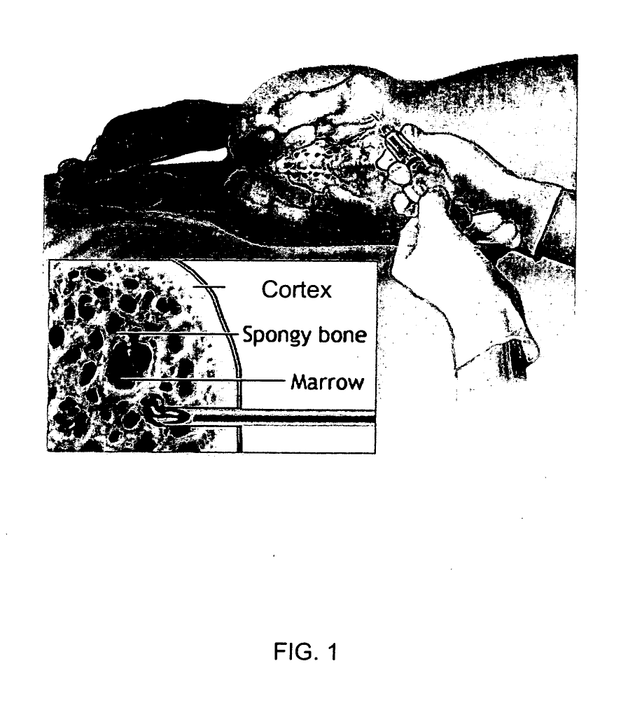

### DETAILED DESCRIPTION

In the following description, numerous specific details are set forth such as examples of specific materials or components in order to provide a thorough understanding of embodiments of the present invention. It will be apparent, however, to one skilled in the art that these specific details need not be employed to practice embodiments of the present invention. In other instances, well known components or methods have not been described in detail in order to avoid unnecessarily obscuring embodiments of the present invention.

The terms “on,” “above,” “below,” “between,” “adjacent,” and “near” as used herein refer to a relative position of one layer or element with respect to other layers or elements. As such, a first element disposed on, above or below another element may be directly in contact with the first element or may have one or more intervening elements. Moreover, one element disposed next to or adjacent another element may be directly in contact with the first element or may have one or more intervening elements.

Any reference to a particular feature, structure, or characteristic described in connection with any one embodiment within this specification should be construed as being included in at least that one embodiment. However, they may also appear in other embodiments, as described in this specification of the claimed subject matter. The appearances of the phrase, “in one embodiment” in various places in the specification are not necessarily all referring to the same embodiment.

Embodiments of a device to aspirate or extract bone marrow tissue from a patient are described. The bone marrow aspiration device described herein facilitates the therapy of autologous bone marrow transplantation in AMI patients by reducing pain associated with the aspiration procedure, improving the efficiency, and tailoring the procedure to the specific requirements of the therapy. In one particular embodiment, the bone marrow aspiration device includes a first outer shaft with a distal cutting tip or edge for penetrating the bone cortex, a proximal handle, and an inner curved, elastic needle with a proximal adaptor suitable for connecting to a syringe. The curved, elastic needle may be made of a shape memory metal such as nickel titanium (NiTi) or another type of super-elastic metal.

In use, the outer shaft is advanced through the bone cortex, for example, of the iliac crest. The curved, elastic needle is introduced into the outer shaft and advanced until the distal end is positioned near the distal end of the outer shaft (i.e., near the distal cutting tip or edge). Suction is applied through a syringe attached near the proximal end of the needle to aspirate bone marrow cells immediately adjacent to the distal cutting tip or edge. After bone marrow cells are obtained from this region, the needle may be advanced further past the distal end of the outer shaft such that the curved end of the needle exits the outer shaft and naturally curves to one side. In one embodiment, the curvature of the needle may be limited to a particular degree (e.g., 90 degrees). The advancement and curvature of the needle allows for a new region of the bone marrow to be accessed for aspiration by device. Aspiration of this new region may be repeated several times by advancing the curved end of the needle in different angular directions and at different depths into the bone marrow containing spongy bone. Thus, the desired amount of bone marrow cells may be aspirated more efficiently and with less pain than with current aspiration systems, which require angular movements of the entire needle, and multiple punctures to obtain the desired amount of bone marrow cells.

In another embodiment of the present invention, the bone marrow aspiration device includes an aspiration needle with a resilient or shape memory wire (e.g., super elastic NiTi wire) disposed within a lumen of the aspiration needle. In use, the aspiration needle is advanced through the cortex and into the bone marrow. Suction is applied to a syringe coupled to the aspiration needle to remove a desired amount of liquid bone marrow. The resilient wire includes a curved portion near the distal end, so that when the distal end is contained within the lumen of the aspiration needle, the curved portion is substantially straight and constrained from curving. When the distal end of the wire is advanced out of the aspiration needle, the curved portion may be used to agitate and disturb the surrounding bone marrow region. This facilitates the aspiration of liquid bone marrow through the aspiration needle. The extent of the wire curvature may be varied and is selected to suit the bone selected for the procedure. In one embodiment, the resilient wire may be moved independent of the aspiration needle. Thus, when the liquid bone marrow is aspirated, the resilient wire may be moved periodically to prevent clogging.

The resilient wire may have various structural configurations. In one embodiment, the resilient wire may have a helical configuration shaped like a corkscrew or simply a single curve bent at an angle. When the resilient wire is rotated on its axis and/or moved in a longitudinal direction, the resilient wire can dislodge clumps of bone marrow tissue or other tissue within the lumen of the aspiration needle to prevent clogging. Alternatively, the resilient wire may be coupled to a mechanical, electrical, or pneumatic actuator to provide vibrational, axial, or rotational movement of the resilient wire. In one embodiment, the resilient wire may be formed by joining a super-elastic shape memory wire (e.g., NiTi) and a high tensile strength wire (e.g., stainless steel) with, for example, a lap joint, or other joints known in the art, to provide a smooth wire surface for the overall resilient wire. The distal portion of the wire may be made of NiTi and the proximal driving portion of the aspiration needle may be made of stainless steel. This configuration provides superior torque and pushability for the aspiration needle to penetrate through the cortex without hindering fluid or cell movement through the aspiration needle.

In another embodiment of the present invention, the bone marrow aspiration device may include a tubular anchoring structure, an aspiration needle which may be disposed within a lumen of the anchoring structure, and a mechanism of engagement between the anchoring structure and the aspiration needle which controls the forward movement of the needle into the bone cortex. Alternatively, the aspiration device may also include a sensor (e.g., torque, pressure, positional) which can monitor the progress of advancing the aspiration needle through the bone cortex.

In use, the anchoring structure is positioned over the outer surface of the bone cortex, allowing the distal end of the anchoring structure to penetrate into the surface of the bone. In one embodiment, the distal end may have external cutting threads, such that with a small amount of rotation and pressure, the distal end can anchor into the surface of the bone cortex. The aspiration needle may then be introduced into the proximal end of the anchoring structure and advanced until the distal tip contacts the cortical surface. The engagement mechanism near the proximal end of the aspiration device may be threads on the inside of the proximal anchoring structure and the outside of the proximal end of the aspiration needle. When the aspiration needle is rotated, a controlled forward movement results, allowing the distal tip of the aspiration needle to penetrate the bone cortex and into the bone marrow cavity. A sensor coupled to the aspiration device may be used to detect when the aspiration needle reaches the bone marrow cavity. Alternatively, the advancement of the aspiration needle may be done in a more controlled manner by a small power unit with a variable speed/torque drive attached near the proximal end of the aspiration device. The drive unit may be coupled to the sensor such that the needle advancement is automatically stopped when the bone cortex is penetrated.

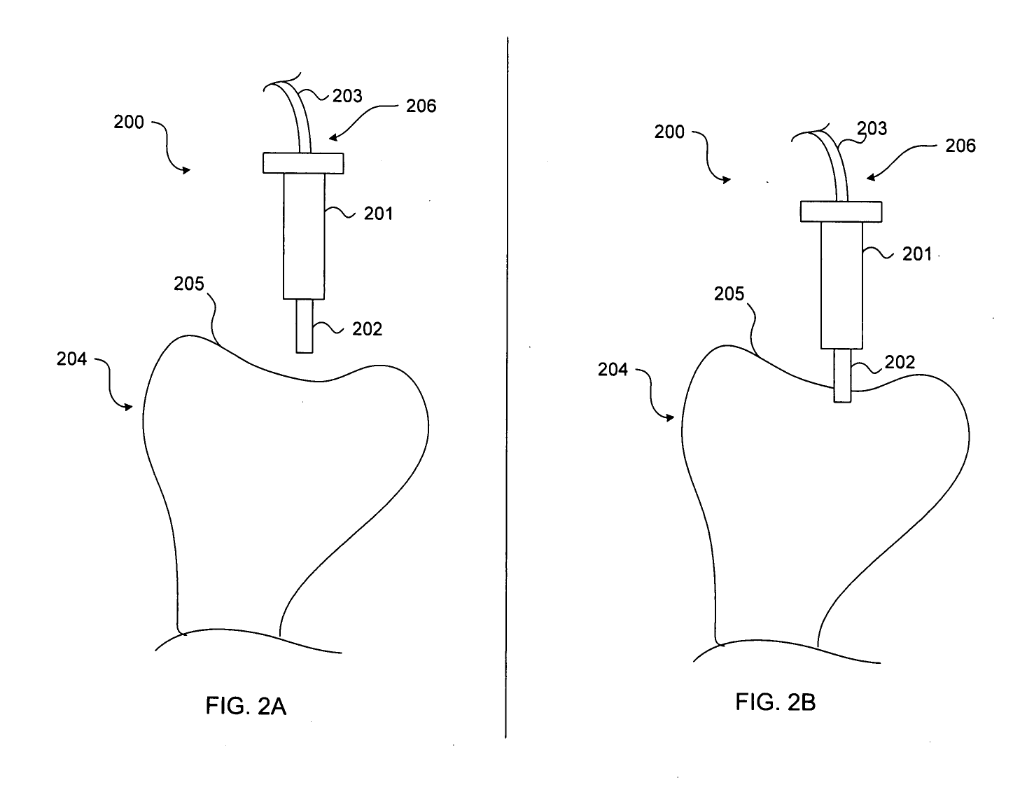

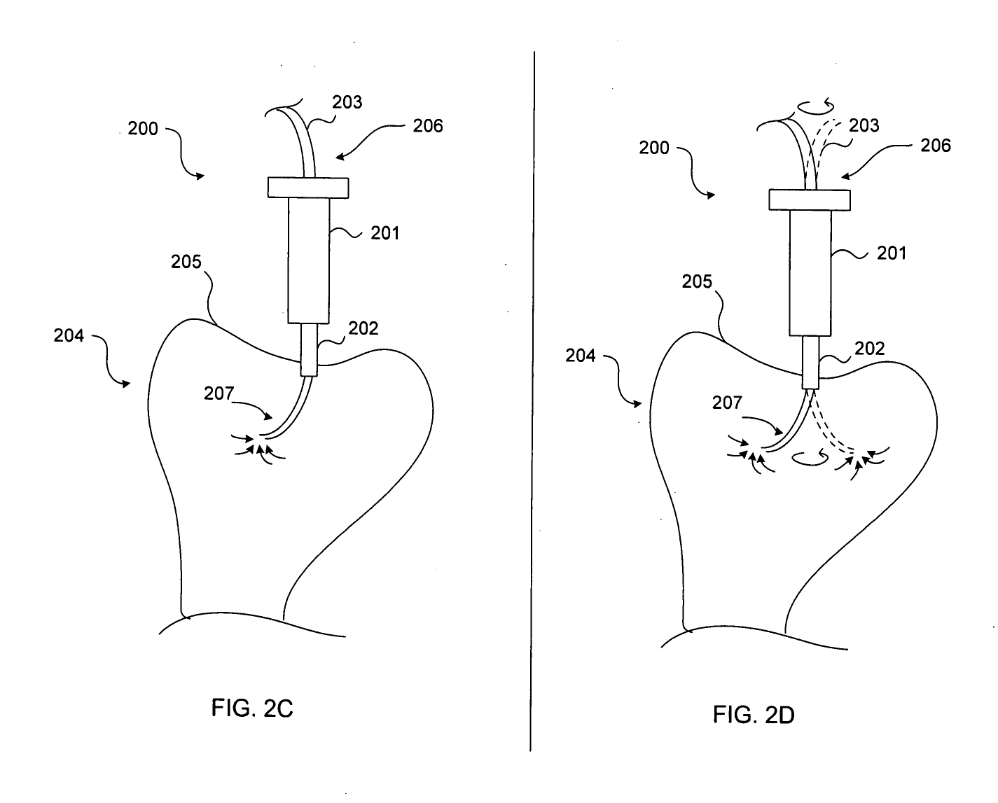

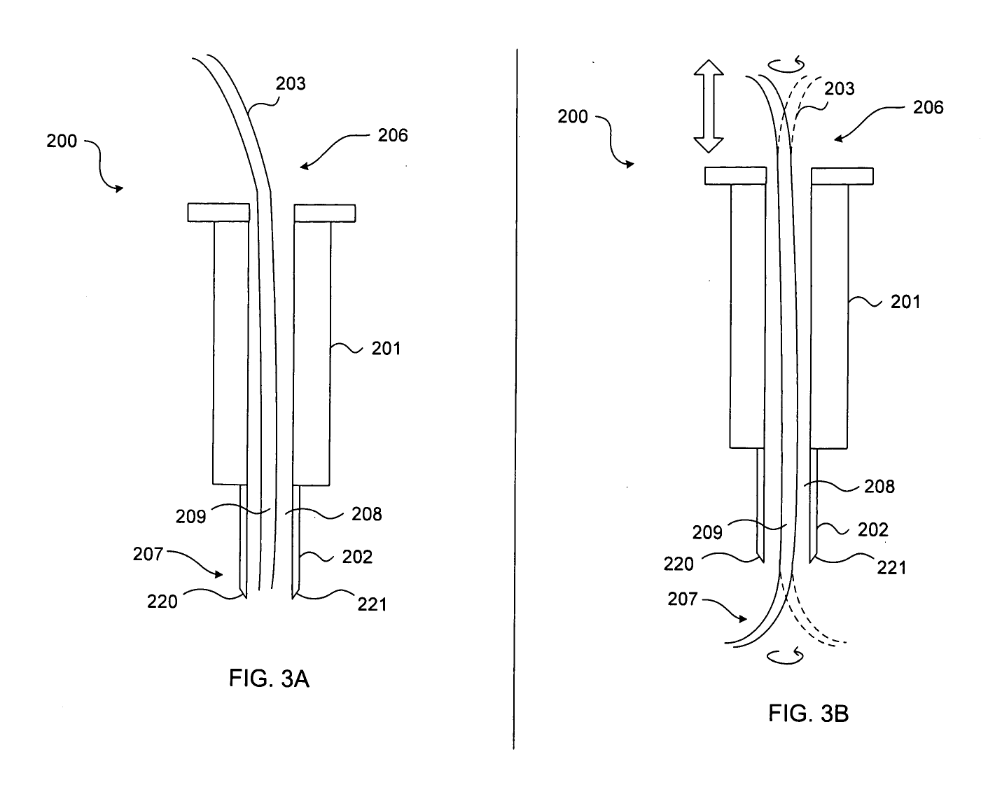

FIGS. 2A-2D illustrate one embodiment of a bone marrow aspiration device 200 and a method to aspirate liquid bone marrow. Bone marrow aspiration device 200 includes an elongated, central body portion 201 and an outer shaft portion 202 coupled near the distal end of the central body portion 201. An aspiration needle 203 may be disposed through an interior lumen of the central body portion 201 and outer shaft portion 202. FIG. 2A illustrates bone marrow aspiration device 200 positioned over a partially illustrated bone structure 204 (e.g., the iliac crest) having bone cortex 205. As illustrated in FIG. 2B, the outer shaft portion 202, which includes a cutting tip or edge (shown in greater detail and described below with respect to FIGS. 3A-3B), penetrates through bone cortex 205 and into the cavity of bone structure 204. Next, as illustrated in FIG. 2C, aspiration needle 203 is advanced through the distal end of outer shaft portion 202 and into the cavity of bone structure 204. A syringe (not shown) may be coupled to aspiration needle near its proximal end to aspirate liquid bone marrow from bone structure 204. Not shown in FIGS. 2C and 2D is that the lumen between the aspiration needle 203 and the outer shaft 202 can be used to inject fluid to make up for marrow volume loss in the bone and to facilitate a continued flow of marrow. On the contrary, this lumen or space can be used to aspirate bone marrow while the needle injects fluid to make up for marrow volume loss in the bone and to facilitate marrow aspiration. This fluid can range from a saline solution, a therapeutic drug that provide an analgesic effect to a biologic solution that promotes faster re-growth of marrow. Specifically, cells or growth factors other than those found in the bone marrow can be used to replace the cells aspirated and in turn stimulate re-growth of marrow. For example, at least one of embryonic stem cells, autologous red blood cells and allogenic red blood cells can be used, in addition to make up fluid such as saline or lactated ringer solution to replace the volume aspirated.

In one embodiment, the proximal portion 206 and the distal portion 207 of aspiration needle 203 may be curved. When extended out from the outer shaft portion 202, the distal portion 207 of aspiration needle 203 curves toward a particular direction as illustrated in FIG. 2C. This allows for a particular region of the bone cavity to be aspirated through aspiration needle 203. When a sufficient amount of liquid bone marrow has been aspirated from a first region, the proximal portion 206 of aspiration needle 203 may be rotated or turned to change the position of distal portion 207, as illustrated in FIG. 2D. This allows the aspiration needle 203 to access a new region of the bone cavity and aspirate additional liquid bone marrow. In an initial aspiration step, the aspiration needle 203 may be advanced past outer shaft 202 without enough of the distal portion 207 exposed for the aspiration needle to curve (e.g., as illustrated in FIG. 2C). This allows for a bone marrow region near the distal end of outer shaft to be aspirated first, before extending aspiration needle 203 to other parts of the bone cavity. In one embodiment, aspiration needle 203 may be moved three-dimensionally, by rotating aspiration needle 203 in a substantially circular manner as well as in an up-and-down direction. Such a needle allows for a maximum number of regions for liquid bone marrow aspiration from a single puncture through the bone cortex 205 of bone structure 204.

FIGS. 3A-3B illustrate sectional views of bone marrow aspiration device 200 in two alternating configurations. In the first configuration of FIG. 3A, the distal portion 207 of aspiration needle 203 is contained within a lumen 208 formed by central body portion 201 and outer shaft 202. The distal portion 207 of aspiration needle 203 maintains a substantially linear shape because it is constrained within lumen 208. When aspiration needle 203 is advanced past the distal opening of outer shaft 202, the natural curvature of the distal portion 207 takes shape as illustrated in FIG. 3B. In one embodiment, the curvature of the distal portion 207 may be up to about 180 degrees relative to the substantially linear portion of aspiration needle 203. Proximal portion 206 of aspiration needle 203 may act as the control handle for controlling the direction of distal portion 207 as well as the distance of distal portion 207 extended past the distal opening of outer shaft 202. The distal tip of outer shaft 202 may have ends 220, 221 shaped as a cutting tip or edge to facilitate the penetration of the outer shaft portion 201 through the bone cortex 205 (as illustrated in FIG. 2C). In one embodiment, ends 220, 221 may be made of a high tensile strength material such as stainless steel. As described above, a syringe (not shown) may be coupled to aspiration needle 203 near proximal portion 206. The syringe may be used to collect the liquid bone marrow aspirated through aspiration needle 203. In an alternative embodiment, aspiration needle 203 may also be used to inject a flushing fluid into the bone cavity. For example, a flushing fluid such as blood or saline may be injected into the puncture site of the bone cortex 205 through a lumen formed by aspiration needle 203. In a slight variation as described above, lumen 208 formed by central body portion 201 and outer shaft 202 can be used to aspirate bone marrow, and the needle 203 which typically may be used to aspirate bone marrow can act in reverse to supply fluid to make up for the bone marrow volume loss from the aspiration. Under general circumstances, the aspiration needle 203 will typically be use to aspirate bone marrow from various location inside the bone while the lumen 208 supplies fluid to replenish the volume loss in the bone. Again, this fluid can range from a saline solution, a therapeutic drug that provide an analgesic effect to a biologic solution that promotes faster re-growth of marrow. Specifically, cells or growth factors other than those found in the bone marrow can be used to replace the cells aspirated and in turn stimulate re-growth of marrow. For example, at least one of embryonic stem cells, autologous red blood cells and allogenic red blood cells can be used, in addition to make up fluid such as saline or lactated ringer solution to replace the volume aspirated.

In one embodiment, aspiration needle 203 may be made of a super-elastic material or metal. In an alternative embodiment, aspiration needle 203 may be made of a shape memory metal such as NiTi. The super-elastic material or shape memory metal of aspiration needle 203 allows for the curvature of distal portion 207 when advanced past the distal opening of outer shaft 202. Aspiration needle 203 may also be made of a combination of super-elastic or NiTi and high tensile strength materials. For example, the distal portion 207 of aspiration needle 203 may be made of NiTi while the rest of aspiration needle 203 is made of stainless steel.

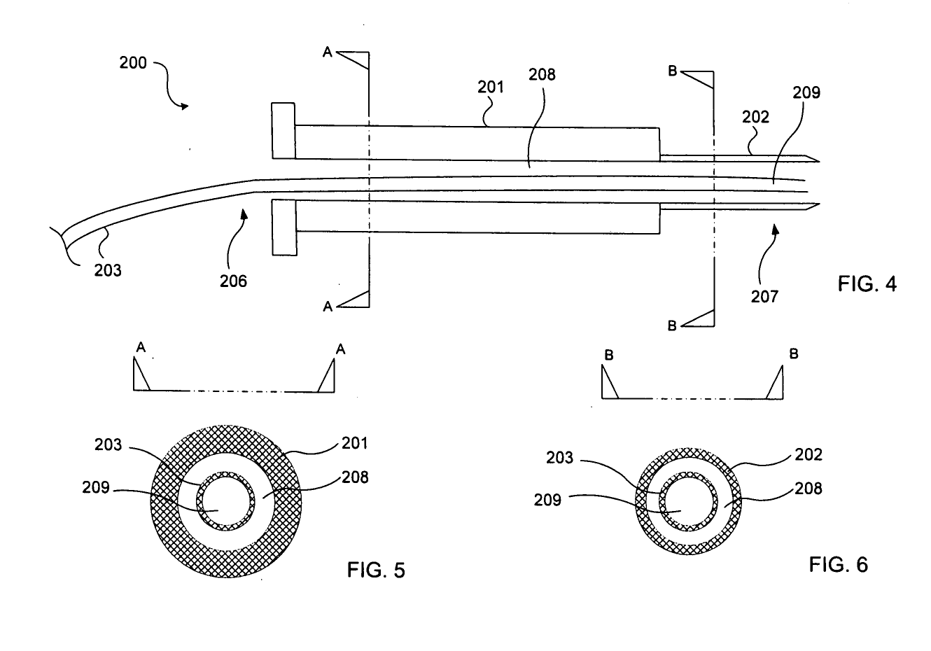

FIG. 4 illustrates another sectional view of bone marrow aspiration device 200, and in particular, illustrating the multiple lumens formed within the device by the aspiration needle 203 and outer shaft 202. A first lumen 208 is formed by central body portion 201 and outer shaft 202. First lumen 208 extends from near the proximal portion 206 of aspiration needle 203 throughout the elongated length of device 200 toward the distal tip of outer shaft 202. Aspiration needle 203 also forms a second lumen 209 for transferring liquid bone marrow through device 200 and into a syringe coupled near the proximal portion 206 of aspiration needle 203.

FIG. 5 illustrates a cross-sectional view of device 200 taken along line A-A and showing first lumen 208 formed by the wall of central body portion 201. Aspiration needle 203 is disposed within first lumen 208, and the wall of aspiration needle 203 forms the second lumen 209. FIG. 6 illustrates a cross sectional view of device 200 taken along line B-B, and showing the wall of outer shaft 202 forming first lumen 208. Aspiration needle 203 is disposed within first lumen 208, and the wall of aspiration needle 203 forms the second lumen 209.

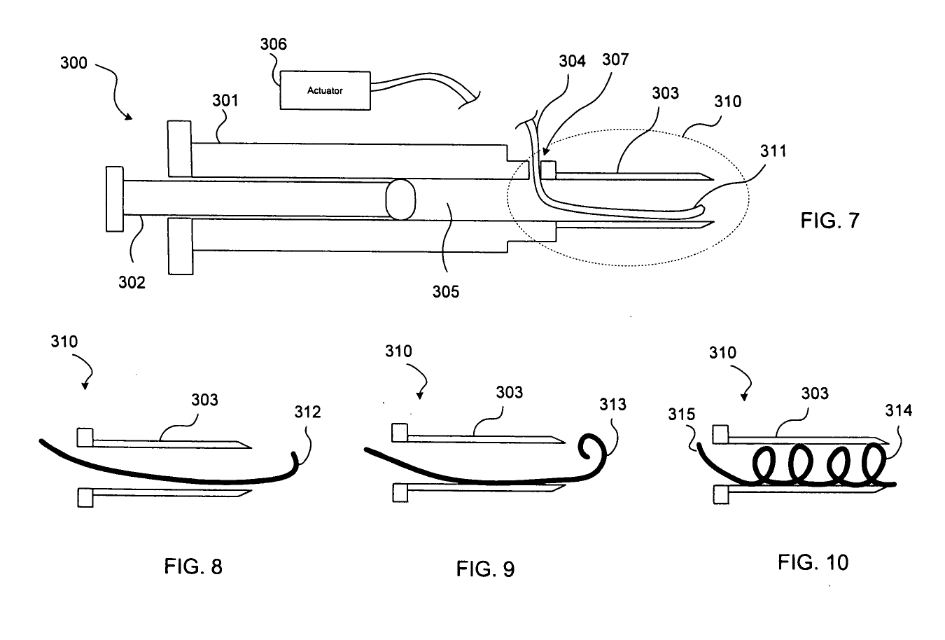

FIG. 7 illustrates a sectional side view of another embodiment of a bone marrow aspiration device 300. A resilient wire 304 disposed within a lumen of device 300 that can agitate or break-up bone marrow tissue to facilitate aspiration. The resilient wire may be driven by an ultrasonic actuator thus using ultrasonic mechanical waves to loosen up bone marrow prior to aspiration. Device 300 includes an elongated syringe body 301 coupled to a needle portion 303 near a distal end. A plunger 302 is disposed within a first lumen 305 formed by the elongated syringe body 301, and first lumen 305 extends along a length of elongated syringe body 301 and needle portion 303. An entry port 307 is formed near a distal end of elongated syringe body 301 to receive resilient wire 304, as illustrated in FIG. 7. Resilient wire 304 extends toward the distal opening of needle portion 303.

In use, the needle portion 303 is advanced through the cortex and into the bone marrow cavity. Suction may be applied device 300 by drawing back plunger 302 so that liquid bone marrow may be collected in lumen 305 formed by elongated syringe body 301. In an alternative embodiment, a separate syringe may be coupled to device 300 to collect the desired amount of liquid bone marrow. Resilient wire 304 includes a curved portion 311 near the distal end, so that when the curved portion 311 is contained within lumen 305 of the needle portion 303, the curved portion 311 is substantially straight and constrained from curving. When the distal end of resilient wire 304 (i.e., curved portion 311) is advanced out of needle portion 303, the curved portion 311 may be used to agitate and disturb the surrounding bone marrow tissue. This facilitates the aspiration of liquid bone marrow through device 300. The extent of the curvature for curved portion 311 may be varied and is selected to suit the bone selected for the procedure. In one embodiment, the resilient wire 304 may be moved independently of the needle portion 303. Thus, when the liquid bone marrow is aspirated, the resilient wire 304 may be moved periodically to prevent clogging.

In one alternative embodiment, resilient wire 304 may be coupled to a mechanical, electrical, or pneumatic actuator (generally represented by actuator 306) to provide vibrational, axial, or rotational movement of resilient wire 304. In one embodiment, the resilient wire 304 may be formed by joining a super-elastic shape memory wire (e.g., NiTi) and a high tensile strength wire (e.g., stainless steel) with a lap joint to provide a smooth wire surface for the overall resilient wire. The curved portion 311 of resilient wire 304 may be made of NiTi and the proximal driving portion of needle portion 303 may be made of stainless steel. This configuration provides superior torque and pushability for needle portion 303 to penetrate through the cortex without hindering fluid or cell movement through device 300.

The resilient wire, and in particular the curved portion, may have various structural configurations. FIGS. 8-10 illustrate isolated views of distal region 310 of device 300 showing alternative embodiments for the curved portion of resilient wire 304. In one embodiment, curved portion 312 may be angled to about 90 degrees as illustrated in FIG. 8. In an alternative embodiment, curved portion 313 may have a coiled end as illustrated in FIG. 9. In another embodiment, the resilient wire 315 disposed within needle portion 303 may have a helical configuration as illustrated in FIG. 10. When rotated on its axis and/or moved in a longitudinal direction, the resilient wire 315 can dislodge clumps of bone marrow tissue or other tissue within lumen 305 to prevent clogging.

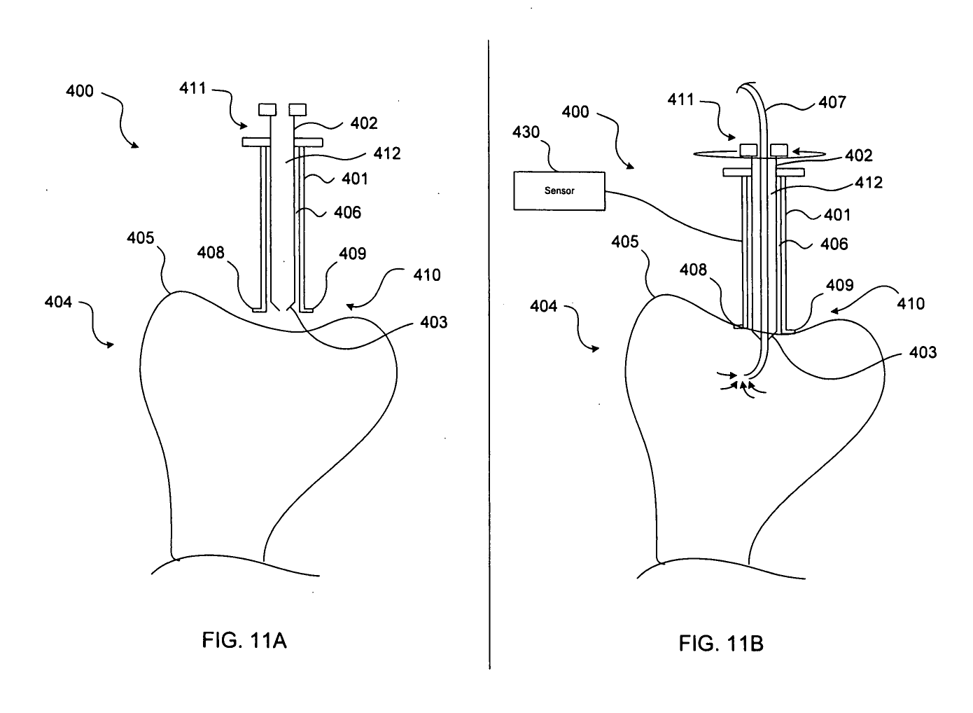

FIGS. 11A-11B illustrate another embodiment of a bone marrow aspiration device 400 and a method to aspirate liquid bone marrow. FIG. 11A illustrates device 400 positioned over a partially illustrated bone structure 404 (e.g., the iliac crest) having bone cortex 405. Bone marrow aspiration device 400 includes an elongated, central body portion 401 and one or more anchoring members 408, 409 disposed near the distal portion 410 of the central body portion 401. In one embodiment, the central body portion 401 of bone marrow aspiration device 400 forms a first lumen 406 to receive a bone penetration needle 402. A proximal portion 411 of bone penetration needle 402 extends out from a proximal end of central body portion 401 and includes a mechanism to drive penetration needle 402 into the bone cavity. The driving mechanism also includes an engagement mechanism for the bone penetration needle 402 and the central body portion 401 (i.e., for the movement of bone penetration needle 402 within lumen 406).

As illustrated in FIG. 11B, anchoring members 408, 409 engage the surface of bone cortex 405 to secure device 400 to bone structure 404. Once secured, the driving mechanism of bone penetration needle 402 is actuated. In one embodiment, the actuation of bone penetration needle 402 may be manually performed, by rotating a dial near proximal portion 411. By rotating the dial, the elongated body of bone penetration needle 402 threads through lumen 406, allowing distal end 403 to advance outward and into bone cortex 405. The rotation of bone penetration needle 402 allows for a controlled forward movement of distal end 403. In an alternative embodiment, the advancement of distal end 403 may be performed in a controlled manner by a small power unit, having a variable speed/torque drive, coupled to bone penetration needle 402 near proximal portion 411. Furthermore, the power unit can incorporate the ability to generate ultrasonic mechanical waves at the distal end of the penetration needle, thus using the ultrasonic frequency vibrations to break up marrow before aspiration. The ultrasonic vibrations can be generated directly by the needle or by a shaped or straight probe through the needle lumen into the bone marrow itself.

In an alternative embodiment, a sensor 430 may be coupled to device 400 to detect a penetration depth for distal end 403 and/or attachment of anchoring members 408, 409 to the surface of bone cortex 405. In another embodiment, sensor 430 may be coupled to the power unit that drives the bone penetration needle 402, such that needle advancement is automatically stopped when bone cortex 405 is penetrated.

Bone marrow tissue may be aspirated from the opening of distal end 403 and through a second lumen 412 of bone penetrating needle 402. For example, a syringe (not shown) may be in fluid communication with second lumen 412 of bone penetrating needle 402 to apply pressure and receive the aspirated liquid bone marrow. In an alternative embodiment, an aspiration needle 407 may be advanced through second lumen 412 of bone penetrating needle 402 and extended distally past the opening of distal end 403. In one embodiment, aspiration needle 407 may be substantially similar to aspiration needle 203 described above with respect to FIGS. 2A-2D and FIGS. 3A-3D. For example, the distal portion of aspiration needle 407 curves toward a particular direction as illustrated in FIG. 11B. The proximal portion of aspiration needle 407 may be coupled to a syringe to collect the liquid bone marrow. This allows for a particular region of the bone cavity to be aspirated through aspiration needle 407. When a sufficient amount of liquid bone marrow has been aspirated from a first region, the aspiration needle 407 may be rotated and moved to another region of the bone cavity for additional aspiration of liquid bone marrow. This allows device 400 to maximize the number of regions within the bone cavity for aspiration.

In one embodiment, aspiration needle 407 may be made of a super-elastic material or metal. In an alternative embodiment, aspiration needle 407 may be made of a shape memory metal such as NiTi. The super-elastic material or shape memory metal of aspiration needle 407 allows for the curvature of the distal portion when advanced past the distal opening of bone penetration needle 402. Aspiration needle 407 may also be made of a combination of super-elastic or NiTi and high tensile strength materials. For example, the distal portion of aspiration needle 407 may be made of NiTi while the rest of aspiration needle 407 is made of stainless steel.

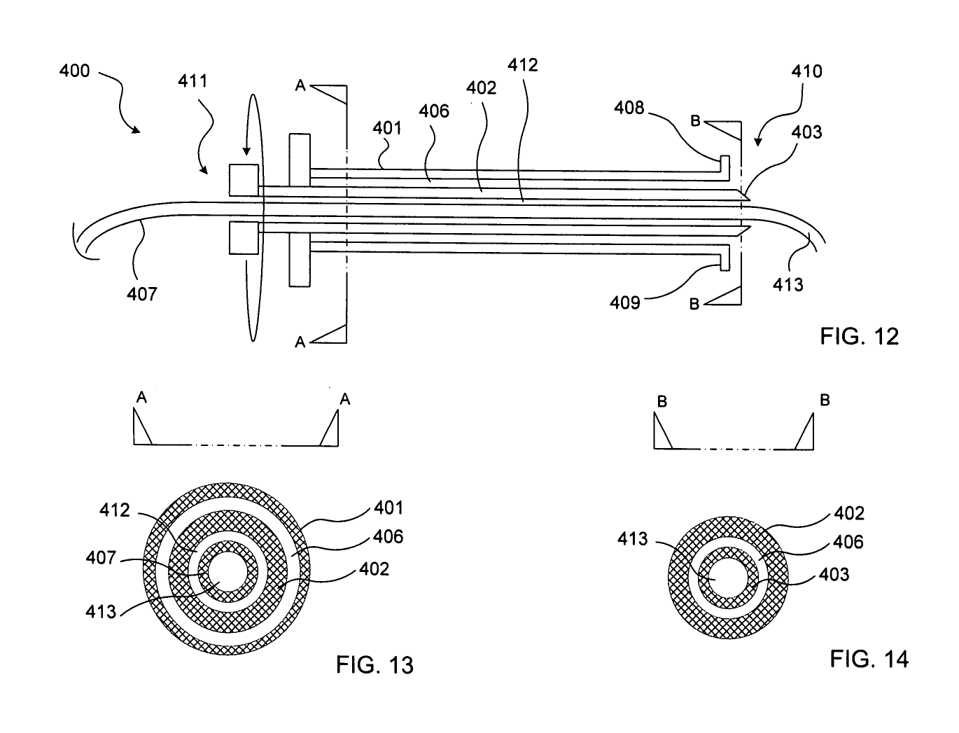

FIG. 12 illustrates a sectional view of bone marrow aspiration device 400, and in particular, illustrating the multiple lumens formed within the device by the central body portion 401, bone penetration needle 402, and aspiration needle 407. A first lumen 406 is formed by central body portion 401, which extends from near the proximal portion 411 of the bone penetration needle 402 throughout the elongated length of device 400 toward anchoring members 408, 409 of distal portion 410. Bone penetration needle 402 also forms a second lumen 412 for transferring liquid bone marrow through device 400 and into a syringe coupled near the proximal portion 411. Alternatively, second lumen 412 may also be used to receive aspiration needle 407. Aspiration needle 407 forms a third lumen 413 which may be used to transfer liquid bone marrow through device 400.

FIG. 13 illustrates a cross-sectional view of device 400 taken along line A-A, which includes central body portion 401, bone penetration needle 402, and aspiration needle 407. A first lumen 406 is formed by the wall of central body portion 401. Bone penetration needle 402 is disposed within first lumen 406, and the wall of bone penetration needle 402 forms the second lumen 412. Aspiration needle 407 is disposed within second lumen 412, and the wall of aspiration needle 407 forms third lumen 413. FIG. 14 illustrates a cross sectional view of device 400 taken along line B-B, which includes bone penetration needle 402 and aspiration needle 407. The wall of bone penetration needle 402 forms second lumen 412. Aspiration needle 407 is disposed within second lumen 412, and the wall of aspiration needle 407 forms the third lumen 413. As described above under FIGS. 2C, 2D, 3A and 3B, the second lumen 412 may be used to infuse fluid to make up for bone marrow volume loss after the aspiration needle lumen 413 aspirated bone marrow from the bone. Conversely, the reverse is possible where lumen 412 is used to aspirate bone marrow, while the needle lumen 413 is used to infuse fluid to make up for volume loss. The fluid injected can range from a saline solution, a therapeutic drug that has an analgesic effect, to a biologic solution that promotes faster re-growth of marrow. Specifically, cells or growth factors other than those found in the bone marrow can be used to replace the cells aspirated and in turn stimulate re-growth of marrow. For example, at least one of embryonic stem cells, autologous red blood cells and allogenic red blood cells can be used, in addition to make up fluid such as saline or lactated ringer solution to replace the volume aspirated.

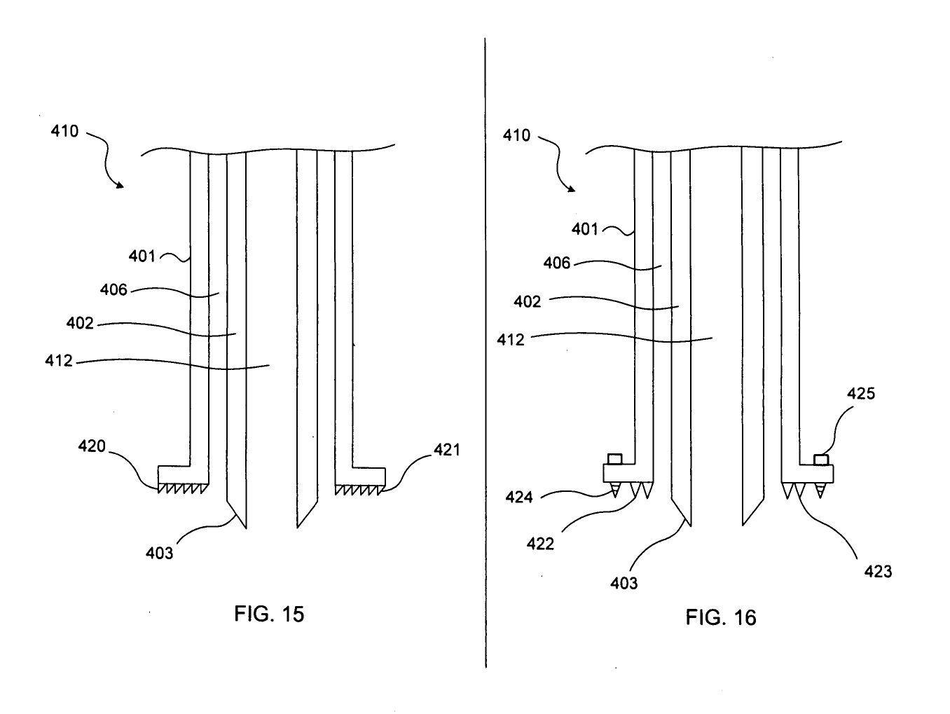

FIGS. 15-16 illustrate sectional views of distal portion 410, showing different embodiments of anchoring members to secure device 400 to the bone cortex 405. Distal portion 410 includes bone penetration needle 402 disposed within first lumen 406 formed by central body portion 401. Distal end 413 of bone penetration needle 402 is advanced distally past the distal opening central body portion 401. For clarity, aspiration needle 407 is not shown (e.g., disposed within second lumen 412 formed by bone penetration needle 402). FIG. 15 illustrates one embodiment of anchoring members 420, 421 having jagged, or teeth-like structures to secure device 400 to the bone cortex (e.g. bone cortex 405). When an operator applies a small amount of pressure against the bone cortex with device 400, anchoring members 420, 421 may penetrate into the surface of the bone cortex. In an alternative embodiment, anchoring members 422, 423 of FIG. 16 may include screws 424, 425 extending through the anchor members to assist the jagged teeth-like structures in anchoring and securing the device onto the cortex of the bone and thus providing stability for the aspiration procedure.

A sensor (e.g., sensor 430) may be coupled to device 400 to detect a penetration depth for distal end 403 and/or attachment of the anchoring members (e.g., 420, 421, 422, 423) to the surface of bone cortex 405. In another embodiment, the sensor may be coupled to a power unit that drives the bone penetration needle 402, such that needle advancement is automatically stopped when the bone cortex is penetrated.

In the foregoing specification, a medical device has been described with reference to specific exemplary embodiments thereof. It will, however, be evident that various modifications and changes may be made thereto without departing from the broader scope of the medical device as set forth in the appended claims. The specification and figures are, accordingly, to be regarded in an illustrative rather than a restrictive sense. Moreover, it is understood that FIGS. 2A-16 are not drawn to scale, and the relative dimensions of the physical structure should not be inferred from the relative dimensions shown in the drawings.

What is claimed:

1. A bone marrow aspiration device, said device comprising:
a central body portion having a proximal end and a distal end;
an outer shaft portion coupled to said distal end of said central body portion, said outer shaft portion having a distal opening;
a first lumen formed by said central body portion and said outer shaft portion, said first lumen extending from said proximal end to said distal opening; and
an aspiration needle disposed within said first lumen, said aspiration needle having a substantially linear configuration when positioned within said first lumen, and a substantially curved configuration when extended from said distal opening, wherein liquid bone marrow can be aspirated through said aspiration needle from a first region of a bone cavity.

2. The device of claim 1, wherein said aspiration needle is rotatable to allow said substantially curved configuration to access a second region of said bone cavity.

3. The device of claim 2, wherein a proximal end of said aspiration needle operates as a controller to rotate said substantially curved configuration.

4. The device of claim 3 further comprises a sensor coupled to the aspiration device to monitor the progress of advancing the aspiration needle through the bone cortex.

5. The device of claim 1, wherein said substantially curved configuration comprises an angle up to 180 degrees relative to said substantially linear configuration.

6. The device of claim 1, wherein said aspiration needle comprises a shape memory metal which includes nickel titanium.

7. The device of claim 1, wherein said aspiration needle forms a second lumen to receive said liquid bone marrow.

8. The device of claim 7, further comprising a syringe coupled to said aspiration needle to collect said liquid bone marrow.

9. The device of claim 1, wherein said distal opening of said outer shaft portion comprises a cutting tip to penetrate through bone cortex and into said bone cavity.

10. The device of claim 9, wherein said cutting tip comprises stainless steel.

11. A bone marrow aspiration device, said device comprising:
a syringe body having a proximal end and a distal end;
an aspiration needle coupled to said syringe body, said aspiration needle having a distal opening;
a first lumen formed by said syringe body and said aspiration needle; and
a resilient wire disposed within first lumen, said resilient wire having a substantially linear configuration when positioned within said first lumen, and a substantially curved configuration when extended from said distal opening, wherein said resilient wire is adapted to contact and break down bone marrow tissue.

12. The device of claim 11, further comprising an entry port disposed on said syringe body to receive said resilient wire.

13. The device of claim 12, wherein said resilient wire is controlled independent of said aspiration needle.

14. The device of claim 11, wherein said aspiration needle is adapted to aspirate liquid bone marrow from said distal opening and through said first lumen into said syringe body.

15. The device of claim 11, wherein said substantially curved configuration of said resilient wire comprises a coiled structure.

16. The device of claim 11, wherein said substantially curved configuration of said resilient wire comprises a helical structure.

17. The device of claim 11, wherein said aspiration needle comprises a shape memory metal, wherein the shape memory metal includes at least nickel titanium.

18. The device of claim 11, wherein said distal opening of said aspiration needle comprises a cutting tip to penetrate through a bone cortex and into said bone cavity.

19. The device of claim 18, wherein said cutting tip comprises stainless steel.

20. The device of claim 11 further comprises a sensor coupled to the aspiration device to monitor the progress of advancing the aspiration needle through the bone cortex.

21. A bone marrow aspiration device, said device comprising:
a central body portion having a proximal end and a distal end, said central body portion to form a first lumen;
a bone penetration needle disposed within said first lumen of said central body portion, said bone penetration needle to form a second lumen; and
a driving mechanism for said bone penetration needle, said driving mechanism to advance said bone penetration needle out of said distal end of said central body portion, wherein said bone penetration needle is adapted to aspirate liquid bone marrow from a bone cavity.

22. The device of claim 21, further comprising an aspiration needle disposed within said second lumen, said aspiration needle having a substantially linear configuration when positioned within said second lumen, and a substantially curved configuration when extended from a distal opening of said bone penetration needle.

23. The device of claim 22, wherein said device further comprises at least one of a torque, pressure, and positional sensor, which can monitor the progress of advancing the aspiration needle through the bone cortex.

24. The device of claim 22, wherein said substantially curved configuration comprises an angle up to 180 degrees relative to said substantially linear configuration.

25. The device of claim 22, wherein said aspiration needle comprises a shape memory metal that includes nickel titanium.

26. The device of claim 22, wherein said aspiration needle is rotatable to allow said substantially curved configuration to access a region of said bone cavity.

27. The device of claim 26, wherein a proximal end of said aspiration needle operates as a controller to rotate said substantially curved configuration.

28. The device of claim 21, wherein the driving mechanism comprises a rotating dial.

29. The device of claim 21, wherein said bone penetration needle is threaded through said first lumen of said central body portion.

30. The device of claim 21, wherein said driving mechanism comprises a motorized driver.

31. The device of claim 30, wherein the driving mechanism is coupled to the sensor such that the needle advancement is automatically stopped when the bone cortex is penetrated.

32. A method for bone marrow aspiration comprising:
inserting into a bone cortex a device with,
a central body having a proximal and a distal end,
an outer shaft portion coupled to said distal end of said central body portion, said outer shaft portion having a distal opening,
a first lumen formed by said central body portion and said outer shaft portion, said first lumen extending from said proximal end to said distal opening, and
at least one of a bone penetration needle and an aspiration needle disposed within said first lumen, said aspiration needle having a substantially linear configuration when positioned within said first lumen, and a substantially curved configuration when extended from said distal opening, wherein said aspiration needle can aspirate liquid bone marrow from a first region of a bone cavity,
penetrating the bone cortex using at least one of a bone penetration needle and a distal cutting tip of a first outer shaft of an aspiration device, and
aspirating bone marrow using at least one of a bone penetration needle and the aspiration needle.

33. The method as in claim 32 wherein said device includes a syringe attached near the proximal end of the aspiration needle to collect said liquid bone marrow and said aspirating is accomplished by suction created in said syringe by drawing back a syringe plunger.

34. The method as in claim 33 wherein said aspiration needle is rotatable to allow said substantially curve configuration to access a second different region of said bone cavity and comprises a shape memory metal which includes nickel titanium.

35. The method as in claim 34 wherein said device further comprises at least one of a torque, pressure and positional sensor which can monitor the progress of advancing the aspiration needle through the bone cortex.

36. The method as in claim 34 wherein said substantially curved configuration comprises an angle up to 180 degrees relative to said substantially linear configuration.

37. The method as in claim 33 wherein said device comprises an entry port disposed on said syringe body to receive said resilient wire.

38. The method as in claim 33 wherein said device contains a resilient wire disposed within a first lumen, said resilient wire having a substantially linear configuration when positioned within said first lumen, and a substantially curved configuration when extended from said distal opening, wherein said resilient wire is adapted to contact and break down bone marrow tissue after initial aspiration of bone marrow from a region.

39. The method as in claim 37 wherein said resilient wire is controlled independent of said aspiration needle.

40. The method as in claim 38 wherein said resilient wire comprises a helical structure and comprising at least one of shape memory metal and shape memory polymer which includes nickel titanium.

41. The method as in claim 32 wherein the bone penetration needle is disposed within said first lumen of said central body portion, advanced out of said distal end of said central body portion by a driving mechanism, and said bone penetration needle can aspirate liquid bone marrow from a bone cavity through a second lumen formed by said bone penetration needle via suction created by a syringe.

42. The method as in claim 41 wherein said driving mechanism comprises at least one of a rotating dial and a motorized driver.

43. The method as in claim 42 wherein said driving mechanism is coupled to a sensor to monitor the progress of advancing the aspiration needle through the bone cortex.

44. The method as in claim 41 wherein said bone penetration needle is threaded through said first lumen of said central body portion.

45. The method as in claim 41 further comprising said aspiration needle disposed within said second lumen, said aspiration needle having a substantially linear configuration when positioned within said second lumen, and a substantially curved configuration when extended from a distal opening of said bone penetration needle.

46. The method as in claim 45 wherein said aspiration needle is rotatable to allow said substantially curve configuration to access a second different region of said bone cavity and comprises a shape memory metal which includes nickel titanium.

47. The method as in claim 46 wherein a proximal end of said aspiration needle operates as a controller to rotate said substantially curved configuration.

48. The method as in claim 47 wherein said substantially curved configuration comprises an angle up to 180 degrees relative to said substantially linear configuration.
